(19) United States
(12) Patent Application Publication
(10) Pub. No.: US 2013/0039967 A1
(43) Pub. Date: Feb. 14, 2013

(54) ORAL DOSAGE FORMS

(71) Applicants: Stephan Meyer, Clarens (CH); Nicole Ouis, Renens (CH)

(72) Inventors: Stephan Meyer, Clarens (CH); Nicole Ouis, Renens (CH)

(73) Assignee: NOVARTIS AG, Basel (CH)

(21) Appl. No.: 13/643,139

(22) PCT Filed: Apr. 19, 2011
(86) PCT No.: PCT/EP11/56257

§ 371 Date: Oct. 24, 2012

(30) Foreign Application Priority Data
Apr. 27, 2010 (EP) 1016161.4

(51) Int. Cl. A61K 9/70; A61P 25/34; A61P 25/26; A61K 31/465

(52) U.S. Cl. 424/443; 514/343

(57) ABSTRACT

The invention relates to specific three layer dosage forms for oral administration of pharmaceutical active substances.

The invention, in particular, concerns a specific oral pharmaceutical dosage form for buccal administration of nicotine representing an edible film and often referred to as thin films or thin strips.

It is well known in the art that using free nicotine base as active in oral dosage forms typically is not convenient because it is known to be very reactive and volatile so that it may not survive the manufacturing process and, in particular, will not be stable in the dosage form over the intended shelf life.

Attempts to obtain a fast disintegrating thin film comprising a nicotine salt (but no pH-raising alkaline substance) failed to provide a product that was able to deliver nicotine to the blood system via transmucosal absorption (see e.g. US 2004/028732 A1). When a thin film was designed by the present inventors that sticked to the buccal mucosa and disintegrated more slowly, e.g. over a period of 3 to 10 minutes, some buccal absorption of nicotine was observed but not to the extent required for the intended purpose, namely for generating a high plasma concentration of nicotine early so as to address nicotine craving symptoms in a patient as soon as possible.

Thus it has been found that when using a nicotine salt as active in a thin strip formulation with the goal of address nicotine craving symptoms early on, typically the addition of an alkaline substance is required to optimize buccal (transmucosal) absorption of nicotine. It has been assessed that the pH in the buccal cavity should be raised to pH 8 or above to optimize the transformation of the nicotine salt (showing low transmucosal absorption only) into buccally well absorbed nicotine free base.

It is therefore an object of the present invention to provide an oral disintegrating film which comprises a pharmaceutically acceptable nicotine salt and an alkaline substance, and which is intended for the administration of nicotine e.g. in smoking cessation therapy.

It was found that, for stability reasons, the alkaline substance could not be added to the same phase as the nicotine salt, because otherwise the latter would quickly be transformed into unstable, volatile nicotine base during manufacture and storage.

Therefore, two layer nicotine thin films were tested in which the nicotine salt and the alkaline substance were physically separated in two different layers that were finally attached to each other. The inventors found, however, that even said two layer nicotine thin films did not show sufficient chemical stability, what could be seen e.g. from a 7% drop of nicotine content after 1 month of storage at 40° C.

Therefore the present invention concerns, in a first preferred embodiment, a pharmaceutical composition in the form of an oral disintegrating film comprising at least three distinct layers (a), (b) and (c),

which first layer (a) comprises a pharmaceutically acceptable salt of nicotine,
which second layer (b) comprises an alkaline substance, and
which third layer (c) is in between layers (a) and (b) thus physically separating them.

The third layer (c) is a separation layer, placed in between layers (a) and (b) to avoid or reduce migration of components from layer (a) to layer (b) and vice versa and, thus, improve the chemical stability of the pharmaceutical dosage form. Due to the presence of the separation layer (c), a pharmaceutically active substance (here: a pharmaceutically acceptable salt of nicotine) is physically separated from a component that is not compatible with said pharmaceutically active substance in the same pharmaceutical form (here: an alkaline substance) during storage. Once the thin film is administered orally, it starts disintegrating in the oral cavity and allows the pharmaceutically acceptable nicotine salt to mix up with the alkaline substance as desired.

In a broader context, the present invention relates to a pharmaceutical composition in the form of an oral disintegrating film comprising at least three distinct layers (a), (b) and (c),

which first layer (a) comprises a pharmaceutically active substance,
which second layer (b) comprises a component that is not compatible with said pharmaceutically active substance of layer (a), and
which third layer (c) is in between layers (a) and (b) thus physically separating them.

“Not compatible with” means “not stable in close contact with each other”, for example because a chemical reaction would take place.

A pharmaceutically active substance of layer (a) can e.g. be an alkaline-labile or acid-labile pharmaceutically active substance, preferably an alkaline-labile pharmaceutically active substance. An alkaline-labile pharmaceutically active substance is, for example, a nicotine component, e.g. a pharmaceutically acceptable salt of nicotine.

In layer (b), a component that is not compatible with the pharmaceutically active substance of layer (a) can e.g. be a second pharmaceutically active substance or an essential excipient. An essential excipient can e.g. be an alkaline substance or an acidic substance, preferably an alkaline substance.

Hereinbefore and hereinafter the term “oral disintegrating film” typically means a single dose unit form which may have any form that is suitable for delivery of the present invention, e.g. strips, rectangles, squares or circles. In the manufacture of said oral disintegrating films, typically three layer laminates are prepared first which are then cut into pieces, e.g. single dose unit forms. Cutting is performed by die-cutting, slitting, laser cutting, or any other technique well known and used in the art.

Returning to the preferred embodiment of the invention, namely a three layer film with a pharmaceutically acceptable salt of nicotine and an alkaline substance being separated, it is preferred that said oral disintegrating films are bio-adhesive, which means that they adhere to the buccal mucosa.

Moreover, it is preferred that said oral disintegrating films completely disintegrate in the mouth of a patient within 1 to 15 minutes—especially 3 to 15 minutes, more especially 3 to 10 minutes and in particular 5 to 8 minutes—after administration to the oral cavity. During said time period, it releases nicotine, formed due the interaction of the pharmaceutically acceptable nicotine salt and the alkaline substance, to the blood stream via the buccal mucosa.

It was found that no particular limitations for the compositions of layers (a), (b) and (c) do exist. Thus, said layers can e.g. be composed of materials known in the art of edible pharmaceutical films and can be manufactured in manner known per se. One or more film-forming polymers typically are the main component of each of the layers. Examples for said film-forming polymers are:

Water soluble film forming polymers, e.g. cellulose, cellulose ether derivatives, synthetically or naturally occurring gums, polyalkylene oxides, polyalkylene glycols; acrylic acid polymers, acrylic acid copolymers, methacrylic acid polymers, methacrylic acid copolymers, polyacrylamides, pullulan, polyvinyl pyrrolidone, polyvinyl alcohol, polyvinyl alcohol copolymers e.g. polyethylene glycol-polyvinyl alcohol copolymers, modified starch, amylose, high amylose starch, hydroxypropylated high amylose starch, dextrin, pectin, chitin, chitosan, levan, elsinan, collagen, gelatin, zein, gluten, soy protein isolate, whey protein isolate or casein.

Non-water soluble film forming polymers are e.g. ethyl cellulose and certain methacrylic acid copolymers, especially copolymers derived from esters—in particular alkyl, aminoalkyl and ammonioalkyl esters—of methacrylic and acrylic acid, e.g. from the Eudragit® series [supplied e.g. by Evonik Roehm GmbH (Darmstadt, Germany)], especially Eudragit® L, S, FS, E, RL or RS polymers (with acidic or alkaline groups) or, in particular, Eudragit® NE polymers (with neutral groups, e.g. methyl or n-butyl ester groups). For selecting non-water soluble film forming polymers, special emphasis is on ethyl cellulose and ethyl acrylate methyl methacrylate copolymers, such as Eudragit® NE 30 D or Eudragit® NE 40 D. Suitable water soluble cellulose ether derivatives include alkyl celluloses e.g. methyl cellulose, substituted alkyl celluloses e.g. hydroxyethyl cellulose, hydroxypropyl cellulose, hydroxypropyl methylcellulose or carboxymethyl cellulose, and salts of substituted alkyl celluloses e.g. sodium carboxymethyl cellulose, and mixtures thereof. Preferred is hydroxypropyl methylcellulose (=HPMC).

Suitable synthetically or naturally occurring gums include xanthan gum, tragacanth gum, guar gum, acacia gum, arabic gum, alginic acid, salts of alginic acid e.g. sodium alginate, dammar gum, gellan gum, gucomannan gum, carrageenan gum, ghatti gum, karaya gum, locust bean gum, tara gum and mixtures thereof. Preferred is xanthan gum.

A polyalkylene oxide is e.g. polyethylene oxide, polypropylene oxide, polybutylene oxide or a copolymer thereof, and in particular polyethylene oxide.

The main criterion for selecting the film-forming polymers, apart from their acceptability in oral pharmaceutical dosage forms, is that a polymer, or a mix of several polymers, is used that meets the desired time for complete disintegration in the mouth of a patient (see above). In cases where thermo-lamination is used to manufacture three-layer films of the invention, the choice of film-forming polymers typically is further influenced by the fact that the temperature applied must be low enough to avoid degradation of any of the components of the three layer laminate to be manufactured, typically 100° C. or less. As a consequence thereof, such film-forming polymers are preferred in thermo-lamination which have a low melting point, e.g. between 40° and 100° C. (see below). It is known to the skilled person in the art how to select film-forming materials that fulfill these requirements.

The thickness of each of the layers (a) and (b) typically is from 10 to 500 micrometers, preferably from 20 to 100 micrometers. The thickness of layer (c) typically is from 3 to 100 micrometers, preferably from 3 to 20 micrometers and in particular from 5 to 15 micrometers.

A pharmaceutically acceptable salt of nicotine is e.g. nicotine bitartrate, nicotine hydrochloride, nicotine dihydrochloride, nicotine citrate or nicotine sulfate, in particular nicotine bitartrate.

An alkaline substance is, for example, sodium carbonate, sodium bicarbonate, potassium carbonate, potassium bicarbonate, sodium hydroxide, potassium hydroxide, or any mixture thereof. Preferred are sodium carbonate and potassium carbonate.

In a particular embodiment of the invention, said alkaline substance is present in layer (b) in suspended form, i.e. not dissolved. This can be accomplished, for example, by using a solvent mixture in the preparation of layer (b) wherein the alkaline substance is not fully soluble, e.g. like isopropanol/water 3:1 to 6:1 in the case of sodium carbonate. By doing so, undesired migration of solubilized alkaline substance towards layer (a) is reduced.

The individual film layers (a), (b) and (c) can be manufactured in any manner known in the art, for example by a casting process or by extrusion, preferably hot melt extrusion. The final three layer films of the invention can be manufactured in a manner known per se, preferably by a lamination process, in particular thermo-lamination. Thermo-lamination means lamination at elevated temperatures, e.g. at 40 to 100° C., especially at 50 to 70° C., in particular 50-60° C. Said elevated temperatures are applied to the layers to be laminated e.g. via heated rolls used in a lamination equipment known in the art.

It is preferred that during the manufacture of the final three layer films the use of water and organic solvents to enable or facilitate the manufacturing process, in particular in the form of a spray, is avoided because the presence of water or organic solvents may favor the undesired migration of components from one layer to another, and moreover, because the presence of organic solvent may give rise to safety concerns (risk of explosions or fire). During manufacture of the individual layers it is fine to use water and organic solvents but before laminating them to the final product, they should typically be dried so that most of the water and organic solvents are removed.

As it typically is desired for thermo-lamination to use moderate heat—e.g. 40 to 100° C., especially 50 to 70° C. and in particular 50-60° C.—and moderate pressure—e.g. 0.2 to 10 kN, especially 0.5 to 2 kN—only, there typically are some requirements for the compositions of the layers in order to be suitable. Therefore, such oral disintegrating films are preferred, wherein the composition of each of the layers (a), (b) and (c) is such that their respective melting points are low enough to allow for a thermo-lamination process without the need of adding water or organic solvent, especially in spray form.

As already discussed above, the separation layer (c) prevents or reduces undesired migration of components from layer (a) to layer (b) and vice versa during storage of the finished product, thus providing stability to edible pharmaceutical films that need to incorporate at least two chemically incompatible substances, such as a nicotine salt and an alkaline substance, e.g. sodium carbonate. After administration of the oral disintegrating film to the oral cavity of a patient, however, it is preferred that the separation layer (c) rapidly disintegrates or dissolves so as to allow fast interaction of two chemically incompatible substances, e.g. a pharmaceutically acceptable salt of nicotine and sodium carbonate, and thereby form the bucally highly absorbable nicotine free base.

Therefore, in a preferred embodiment of invention, the separation layer (c) is thinner than each of the layers (a) and (b), e.g. having a thickness of 20 micrometers or less, e.g. 3-20—preferably 5-15 and in particular 5-10—micrometers.

The beneficial properties of the pharmaceutical compositions of the invention are demonstrated e.g. by the following tests:

The stability of three layer films as disclosed in Examples 6b and 7 has been tested in comparison with corresponding bi-layer product obtained by direct lamination of a layer of Example 1 on to a layer of Example 2. Very challenging (so-called “stress”) conditions, namely a temperature of T=40° C. (and 75% relative humidity) were applied over a period of 14 weeks. In contrast to the comparative bi-layer product, so significant drop of the nicotine content was observed with the three layer films of Examples 6b and 7.

Stability under stress conditions (at 40°
C. and 75% relative humidity after 14 weeks)

| | | Σ Nicotine degradation |
|---|---|---|
| Example: Layers laminated | Nicotine loss (%) | products (%) |
| Comparison: Ex 1 + Ex 2 | 27.9 | 5.0 |
| Ex 6b: Ex 1 + Ex 4 + Ex 2 | 9.9 | 3.1 |
| Ex 7: Ex 1 + Ex 5 + Ex 2 | 4.8 | 3.2 |

After oral administration of a three layer film as disclosed in Example 6, nicotine plasma concentrations at various time points are measured and corresponding AUCs (AUC=area under curve) at various time points [especially AUC (0-5 min), AUC (0-10 min) and AUC (0-20 min)] derived from the corresponding pharmacokinetic curves.

The following examples are intended to illustrate the invention. All amounts indicated are given in mg.

### EXAMPLE 1

### Layer Comprising Sodium Carbonate

| Ingredients | Amount (mg) |
|---|---|
| Polyvinylpyrrolidone (PVP) | 24.00 |
| Hydroxypropyl methyl cellulose (HPMC) | 9.00 |
| Ethyl cellulose | 10.00 |
| Polyethylene oxide (POLYOX 80) | 3.00 |
| Polyethylene glycol 400 | 5.00 |
| Sodium carbonate (250 micron sieve/screen) | 9.00 |
| Microcrystalline cellulose | 10.00 |
| Titanium dioxide | 1.00 |
| Levomenthol | 1.50 |
| Acesulfame K | 0.50 |
| Spearmint flavor | 0.50 |
| Isopropanol | 165.00 |
| Water, purified | 35.85 |
| Total wet mass | 274.35 |
| Total dry mass | 73.50 |

Process: Sieve sodium carbonate on a 250 micron screen. Disperse or dissolve sodium carbonate, microcrystalline cellulose, titanium dioxide, levomenthol and Acesulfame K in a mixture of purified water, isopropanol, spearmint flavor and polyethylene glycol 400. Then, disperse polyvinylpyrrolidone, hydroxypropyl methyl cellulose, ethyl cellulose and polyethylene oxide in said mixture. Mix until homogeneity. Once the mix is homogeneous, it is casted on a liner and dried in an oven in a discontinuous process.

### EXAMPLE 2

### Layer Comprising Nicotine Bitartrate

| Ingredients | Amount (mg) |
|---|---|
| Polyvinylpyrrolidone | 24.00 |
| Hydroxypropyl methyl cellulose | 9.00 |
| Ethyl cellulose | 10.00 |
| Polyethylene oxide (POLYOX 80) | 3.00 |
| Polyethylene glycol 400 | 5.00 |
| FD&C Blue No. 1 (colorant) | 0.03 |
| Microcrystalline cellulose | 19.00 |
| Titanium dioxide | 1.00 |
| Levomenthol | 1.50 |
| Acesulfame K | 0.50 |
| Spearmint flavor | 0.50 |
| Nicotine bitartrate dihydrate | 3.07 |
| Isopropanol | 165.00 |
| Water, purified | 35.85 |
| Total wet mass | 277.45 |
| Total dry mass | 76.60 |

Process: Disperse or dissolve nicotine bitartrate dihydrate, the colorant, microcrystalline cellulose, titanium dioxide, levomenthol and Acesulfame K in a mixture of purified water, isopropanol, spearmint flavor and polyethylene glycol 400. Then, disperse polyvinylpyrrolidone, hydroxypropyl methyl cellulose, ethyl cellulose and polyethylene oxide in said mixture. Mix until homogeneity. Once the mix is homogeneous, it is casted on a liner and dried in an oven in a discontinuous process.

### EXAMPLE 3

### Disintegrable Separation Layer (c)

| Ingredients | Amount (mg) |
|---|---|
| Ethyl acrylate/methyl methacrylate copolymer | 10.00 |
| (Eudragit NE 30 D) | |
| Methacrylic acid/methyl methacrylate copolymer | 15.00 |
| (Eudragit L) | |
| Aceton | 5.00 |
| Isopropanol | 20.00 |
| Total wet mass | 50.00 |
| Total dry mass | 25.00 |

Process: Eudragit NE 30 D and Eudragit L are dissolved in a mixture of isopropanol and acetone and mixed until homogeneity.

### EXAMPLE 4

### Disintegrable Separation Layer (c)

| Ingredients | Amount (mg) |
|---|---|
| Methacrylic acid/methyl methacrylate copolymer | 7.50 |
| (Eudragit S) | |
| Methacrylic acid/methyl methacrylate copolymer | 15.50 |
| (Eudragit L) | |
| Aceton | 5.00 |
| Isopropanol | 50.00 |
| Total wet mass | 78.00 |
| Total dry mass | 23.00 |

Process: analogous to Example 3.

### EXAMPLE 5

### Soluble Separation Layer (c)

| Ingredients | Amount (mg) |
|---|---|
| Polyethylene oxide (POLYOX 80) | 6.00 |
| Glycerol | 0.25 |
| Water, purified | 9.00 |
| Isopropanol | 21.00 |
| Total wet mass | 36.25 |
| Total dry mass | 6.25 |

Process: Glycerol and polyethylene oxide are dissolved or dispersed, respectively in a mixture of isopropanol and water and mixed to homogeneity.

### EXAMPLE 6a

### Thermo-Lamination of the Layers of Examples 1, 2 and 3

The layer of Example 1 is attached on one side to a casting liner and the other side is exposed. The layer of Example 3 is similarly attached on one side to a casting liner and the other side is exposed. The two exposed sides of Examples 1 and 3 are pressed against each other by feeding the layers through two lamination rolls which are heated to 60° C. The casting liner of the “Example 3 layer” is removed, thereby exposing the side which has not been laminated to the layer of Example 1. The layer of Example 2 is attached on one side to a casting liner and the other side is exposed. Its exposed side is pressed against the newly exposed side of the “Example 3 layer” within the laminate “Example 1 layer/Example 3 layer” prepared before. This is again done between two heated lamination rolls which are heated to 60° C. A three layer laminate of “Ex1/Ex3/Ex2 layers” is obtained.

### EXAMPLE 6b

### Thermo-Lamination of the Layers of Examples 1, 2 and 4

Example 6a is repeated but instead of the layer of Example 3 the layer of Example 4 is used. A three layer laminate of “Ex1/Ex4/Ex2 layers” is obtained.

### EXAMPLE 7

### Thermo-Lamination (with Spraying Ethanol) of the Layers of Examples 1, 2 and 5

The layer of Example 1 is attached on one side to a casting liner and the other side is exposed. The layer of Example 5 is similarly attached on one side to a casting liner and the other side is exposed. The two exposed sides of Examples 1 and 5 are pressed against each other, while spraying ethanol, by feeding the layers through two lamination rolls which are heated to 60° C. The casting liner of the “Example 5 layer” is removed, thereby exposing the side which has not been laminated to the layer of Example 1. The layer of Example 2 is attached on one side to a casting liner and the other side is exposed. Its exposed side is pressed against the newly exposed side of the “Example 5 layer” within the laminate “Example 1 layer/Example 5 layer” prepared before. This is again done between two heated lamination rolls which are heated to 60° C. A three layer laminate of “Ex1/Ex5/Ex2 layers” is obtained.

### EXAMPLE 8

Each of the three layer laminates obtained in Examples 6a, 6b and 7 is cut via die-cutting into rectangular pieces comprising 3.07 mg of nicotine bitartrate dihydrate and 9.00 mg sodium carbonate.

1. A pharmaceutical composition in the form of an oral disintegrating film comprising at least three distinct layers (a), (b) and (c),
which first layer (a) comprises a pharmaceutically active substance,
which second layer (b) comprises a component that is not compatible with said pharmaceutically active substance of layer (a), and
which third layer (c) is in between layers (a) and (b) thus physically separating them.

2. A pharmaceutical composition according to claim 1 wherein said pharmaceutically active substance of layer (a) is an alkaline-labile or acid-labile pharmaceutically active substance.

3. A pharmaceutical composition according to claim 2, wherein said pharmaceutically active substance of layer (a) is a nicotine salt.

4. A pharmaceutical composition according to claim 1, wherein said component that is not compatible with the pharmaceutically active substance of layer (a) is either a second pharmaceutically active substance or an essential excipient selected from the group consisting of alkaline substances and acidic substances.

5. A pharmaceutical composition according to claim 1 in the form of an oral disintegrating film comprising at least three distinct layers (a), (b) and (c),
which first layer (a) comprises a pharmaceutically acceptable salt of nicotine,
which second layer (b) comprises an alkaline substance, and
which third layer (c) is in between layers (a) and (b) thus physically separating them.

6. A pharmaceutical composition according to claim 5, wherein said alkaline substance is selected from the group consisting of sodium carbonate, sodium bicarbonate, potassium carbonate, potassium bicarbonate, sodium hydroxide, potassium hydroxide, or any mixture thereof.

7. A pharmaceutical composition according to claim 6, wherein said alkaline substance is present in layer (b) in suspended form.

8. A pharmaceutical composition according to claim 5, wherein said third layer (c) has a thickness of 3-20 micrometers, preferably 5-15 micrometers.

9. A pharmaceutical composition according to claim 1, which is bio-adhesive and completely disintegrates within 3 to 15 minutes, preferably 5 to 8 minutes, after administration to the oral cavity.

10. A pharmaceutical composition according to claim 1, wherein the compositions of each of the layers (a), (b) and (c) are such that their respective melting points are low enough to allow for a thermo-lamination process without the need of adding water or organic solvent.

11. A pharmaceutical composition according to claim 10, wherein said thermo-lamination process uses heat of from 40 to 100° C. and moderate pressure of 0.2 to 10 kN.

12. A pharmaceutical composition according to claim 1, wherein both layers (a) and (b) comprise polyvinyl pyrrolidone and hydroxypropyl methyl cellulose.

13. A pharmaceutical composition according to claim 12, wherein both layers (a) and (b) in addition also comprise ethyl cellulose.

14. A pharmaceutical composition according to claim 12, wherein both layers (a) and (b) in addition further comprise polyethylene oxide.

15. A pharmaceutical composition according to claim 1, wherein layer (c) comprises at least one polymer selected from the group consisting of an ethyl acrylate/methyl methacrylate copolymer, a methacrylic acid/methyl methacrylate copolymer, a methacrylic acid/ethyl acrylate copolymer and a polyethylene oxide.

16. A pharmaceutical composition according to claim 3, wherein said component that is not compatible with the pharmaceutically active substance of layer (a) is either a second pharmaceutically active substance or an essential excipient selected from the group consisting of alkaline substances and acidic substances.

17. A pharmaceutical composition according to claim 5, which is bio-adhesive and completely disintegrates within 3 to 15 minutes, preferably 5 to 8 minutes, after administration to the oral cavity.

18. A pharmaceutical composition according to claim 21, which is bio-adhesive and completely disintegrates within 3 to 15 minutes, preferably 5 to 8 minutes, after administration to the oral cavity.

19. A pharmaceutical composition according to claim 6, wherein layer (c) comprises at least one polymer selected from the group consisting of an ethyl acrylate/methyl methacrylate copolymer, a methacrylic acid/methyl methacrylate copolymer, a methacrylic acid/ethyl acrylate copolymer and a polyethylene oxide.

20. A pharmaceutical composition according to claim 12, wherein layer (c) comprises at least one polymer selected from the group consisting of an ethyl acrylate/methyl methacrylate copolymer, a methacrylic acid/methyl methacrylate copolymer, a methacrylic acid/ethyl acrylate copolymer and a polyethylene oxide.

21. A pharmaceutical composition according to claim 1, wherein
first layer (a) comprises a pharmaceutically acceptable salt of nicotine,
second layer (b) comprises an alkaline substance selected from the group consisting of sodium carbonate, sodium bicarbonate, potassium carbonate, potassium bicarbonate, sodium hydroxide, potassium hydroxide, or any mixture thereof, and
third layer (c) comprises at least one polymer selected from the group consisting of an ethyl acrylate/methyl methacrylate copolymer, a methacrylic acid/methyl methacrylate copolymer, a methacrylic acid/ethyl acrylate copolymer and a polyethylene oxide,
wherein both layers (a) and (b) comprise polyvinyl pyrrolidone and hydroxypropyl methyl cellulose, and
wherein the compositions of each of the layers (a), (b) and (c) are such that their respective melting points are low enough to allow for a thermo-lamination process without the need of adding water or organic solvent.

22. A composition according to claim 21, wherein layers (a) and (b) also comprise ethyl cellulose and polyethylene oxide.
